(19) United States
(12) Patent Application Publication
(10) Pub. No.: US 2009/0287205 A1
(43) Pub. Date: Nov. 19, 2009

(54) SYSTEMS AND METHODS FOR PREVENTING TISSUE POPPING CAUSED BY BUBBLE EXPANSION DURING TISSUE ABLATION

(75) Inventor: Frank Ingle, Palo Alto, CA (US)

Correspondence Address: VISTA IP LAW GROUP LLP, 12930 Saratoga Avenue, Suite D-2, Saratoga, CA, 95070, US

(73) Assignee: BOSTON SCIENTIFIC SCIMED, INC., Maple Grove, MN (US)

(21) Appl. No.: 12/463,904

(22) Filed: May 11, 2009

Related U.S. Application Data
(60) Provisional application No. 61/054,066, filed on May 16, 2008.

(51) Int. Cl. A61B 18/18; A61B 18/12

(52) U.S. Cl. 606/33; 606/34

(57) ABSTRACT

A system for controllably delivering ablation energy to tissue includes an ablation device operable to supply ablation energy to body tissue causing bubbles to form in the tissue, an ultrasound transducer configured to detect energy spontaneously emitted by collapsing or shrinking bubbles that are resonating in the tissue, and a control element operably coupled to the ablation device and the ultrasound transducer element, the control element being configured to adjust the ablation energy supplied to the tissue in response to the energy detected by the ultrasound transducer to prevent tissue popping caused by bubble expansion.

### RELATED APPLICATION DATA

The present application claims the benefit under 35 U.S.C. §119 to U.S. provisional patent application No. 61/054,066, filed May 16, 2008. The foregoing application is hereby incorporated by reference into the present application in its entirety.

### FIELD OF THE INVENTION

The present inventions relate generally to controlling electro-surgical probes and devices that are used for tissue ablation.

### BACKGROUND

It is known to ablate tissue using various ablation instruments for treatment of medical conditions, e.g., to treat cardiac fibrillation and other conditions. One known problem associated with known ablation devices involves overheating of tissue, which may result in audible “tissue popping.” In these cases, tissue popping results from formation of bubbles within heated tissue when the tissue is heated and expansion, explosion or rupturing of tissue by these bubbles. The resulting “popping” sounds may loud enough such that they are heard by a physician and patient.

Bubble formation and expansion and subsequent popping present a number of shortcomings and undesirable effects. For example, tissue popping caused by expansion of bubbles may result in tearing of tissue and release of emboli or micro-bubbles into the blood. This may cause more serious negative consequences including, for example, interruption of blood flow to tissue, distal circulatory damage and stroke. Moreover, a conscious patient may hear his or her own tissue “popping” as a result of bubbles expanding and exploding during an ablation procedure. The patient may be stressed or disturbed upon hearing these popping sounds.

Present clinical practices and known devices, however, do not effectively prevent tissue popping due to bubble expansion and/or require tissue popping by bubble expansion in order to determine that ablation energy levels should be reduced. For example, certain clinical practices rely on actually hearing tissue popping sounds that are caused by bubble expansion in response to which a physician may reduce the amount of ablation energy that is applied to tissue. Other systems do not rely on the ear of a physician and instead include detection mechanism that detects sounds generated by tissue popping caused by bubble expansion, in response to which ablation energy may be reduced. However, in both cases, the control mechanisms rely on tissue popping by bubble expansion to occur and, therefore, rely on a detection process that involves associated tissue damage, release of emboli into the blood and other negative effects.

### SUMMARY

Embodiments are directed to ablation devices and methods that deliver ablation energy to an ablation device, such as a catheter and other suitable ablation devices, in a controlled manner to prevent tissue rupture caused by expansion of bubbles in heated tissue, otherwise referred to as tissue popping caused by bubble expansion.

One embodiment is directed to a system for controllably delivering ablation energy to tissue. The system comprises an ablation device, an ultrasound transducer, and a control element (e.g., processor, hardware, software or computer). The ablation device is configured to supply ablative energy to tissue, thereby resulting in the formation of bubbles in the tissue. The transducer element is configured to detect energy spontaneously emitted by collapsing or shrinking bubbles that resonate within the tissue. The control element is operably coupled to the ablation device and the transducer element and configured to adjust ablation energy supplied to tissue in response to the detected energy, e.g., in response to an amplitude of the detected energy, in order to prevent tissue popping caused by bubble expansion.

In another embodiment, a system for controllably delivering ablation energy to tissue comprises an ablation device, first and second transducers, and a control element. The ablation device is configured to supply ablation energy to tissue, thereby resulting in the formation of bubbles within the tissue. A first transducer element is configured to insonate tissue undergoing ablation with an interrogation signal, and a second transducer element is configured to detect energy emitted by collapsing or shrinking bubbles that resonate within the tissue in response to the interrogation signal. The control element is operably coupled to the ablation device and the second transducer element and configured to adjust the ablation energy supplied to tissue in response to the detected energy, e.g., in response to an amplitude of the detected energy, in order to prevent tissue popping caused by bubble expansion.

A further embodiment is directed to a method of controllably ablating tissue. The method comprises applying ablation energy to tissue, thereby forming bubbles within tissue, detecting ultrasound energy spontaneously emitted by collapsing bubbles resonating within tissue and adjusting the ablation energy applied to tissue in response to the detected energy, e.g., in response to an amplitude of the detected energy, in order to prevent tissue popping caused by bubble expansion.

A further embodiment is directed to a method of controllably ablating tissue using a plurality of transducer elements. The method comprises applying ablation energy to tissue, thereby forming bubbles within tissue, insonating tissue with an ultrasound interrogation signal emitted by a first transducer element, detecting an energy emitted by collapsing bubbles resonating within the tissue in response to the ultrasound interrogation signal, and adjusting ablation energy provided to tissue in response to the detected energy, e.g., in response to an amplitude of the detected energy, in order to prevent tissue rupture caused by bubble expansion.

In one or more embodiments, the ablation device is a radio frequency ablation device and embodiments may be implemented using or incorporated within an ablation catheter. Thus, embodiments can be implemented such that the ablation device is an ablation device other than a high intensity focused ultrasound ablation device.

In one or more embodiments, a transducer element is configured to detect energy spontaneously emitted by a bubble at a resonant frequency that is based on a size of the bubble. Further, ablation energy can be adjusted to maintain bubble diameters less than about 100 micrometers to prevent tissue popping by bubble expansion.

Embodiments can be implemented using a single frequency or multi-frequency interrogation signal. The interrogation signal may also be a band limited spread spectrum signal. Further, the interrogation signal is a frequency hopping signal. In one or more embodiments, the energy emitted by the bubbles includes a plurality of harmonics or sub-harmonics of a frequency of an interrogation signal.

### BRIEF DESCRIPTION OF DRAWINGS

Embodiments will be described and explained with additional specificity and detail through the use of the accompanying drawings in which:

FIG. 1 illustrates a system constructed according to one embodiment that includes a single transducer element configured to detect energy spontaneously emitted by bubbles to prevent tissue popping by bubble expansion;

FIG. 2 illustrates a system constructed according to another embodiment that includes multiple transducer elements and that is configured to controllably deliver energy to an ablation device to prevent tissue popping by bubble expansion based on detection of harmonics and sub-harmonics of an interrogation signal;

FIG. 2A is a graph illustrating a spectrum of an interrogation signal emitted by a transducer element shown in FIG. 2;

FIG. 2B is a graph illustrating a spectrum of frequencies of energy emitted by bubbles in tissue exposed to the interrogation signal shown in FIG. 2A;

FIG. 2C is a graph illustrating a spectrum of a band reject filtered or “notched out” signal for removing a frequency of the interrogation signal from energy emitted by bubbles;

FIG. 2D is a graph illustrating a spectrum of a signal that is reflected from a surface or area that does not include any bubbles in response to an interrogation signal;

FIG. 3 illustrates a system constructed according to another embodiment that is configured to controllably deliver energy to an ablation device while preventing tissue popping by bubble expansion utilizing sub-harmonic and harmonic detection and mixing;

FIG. 3A is a graph illustrating a fixed clock signal that may be utilized with the system shown in FIG. 3;

FIG. 3B is a graph illustrating a spectrum of frequencies of energy emitted by bubbles in the system shown in FIG. 3;

FIG. 3C is a graph illustrating a spectrum of a mixed signal that is based on the signal shown in FIG. 3B and that may be utilized in the system shown in FIG. 3;

FIG. 3D illustrates a spectrum of a band-pass filter that passes frequencies from f₀/2 to 3f₀ and that may be used with the system shown in FIG. 3;

FIG. 4 illustrates a system constructed according to yet another embodiment that employs frequency hopping and that is configured to controllably deliver energy to an ablation device while preventing tissue popping;

FIG. 4A is a graph illustrating a drive signal at a first frequency f₀;

FIG. 4B is a graph illustrating a drive signal at a different frequency f₁;

FIG. 5 illustrates a system constructed to another embodiment that employs band limited white noise for an ultrasonic interrogation signal and that is configured to controllably deliver energy to an ablation device to prevent tissue popping caused by bubble expansion;

FIG. 5A is a graph showing a spectrum of energy emitted by bubbles using the system shown in FIG. 5 and a harmonic and sub-harmonics;

FIG. 6 illustrates a system constructed according to another alternative embodiment that utilizes periodic impulses for interrogation signals and that is configured to controllably deliver energy to an ablation device to prevent tissue popping caused by bubble expansion;

FIG. 6A is a graph showing an example of energy emitted by three representative bubbles;

FIG. 6B is a graph showing a transition of a range gate from an off state to an on state for use in the embodiment shown in FIGS. 6 and 6A;

FIG. 7 illustrates a system constructed according to one embodiment that includes an ablation device in the form of an ablation catheter in which embodiments may be implemented;

FIG. 7A illustrates one manner in which embodiments may be implemented within an ablation device in the form of an ablation catheter in which a transducer is electrically connected to a source of radio frequency energy;

FIG. 7B illustrates one manner in which embodiments may be implemented within an ablation device in the form of an ablation catheter in which a transducer is insulated from a source of radio frequency energy;

FIG. 7C illustrates another manner in which embodiments may be implemented within an ablation catheter that includes a solid ablation tip and a flat disc-shaped ultrasound transducer;

FIG. 7D illustrates yet another manner in which embodiments may be implemented in a closed or open irrigated ablation catheter;

FIG. 7E illustrates another manner in which embodiments may be implemented in an ablation device that includes a solid ablation tip and two flat half-disc-shaped ultrasound transducers for transmitting and receiving energy;

FIG. 8 illustrates a system constructed according to yet another embodiment that utilizes a single ultrasonic transducer and is configured to controllably deliver energy to an ablation device to prevent tissue popping by bubble expansion; and

FIGS. 8A and 8B illustrate advantages of embodiments that utilize a single transducer element as shown in FIG. 8.

### DETAILED DESCRIPTION OF ILLUSTRATED EMBODIMENTS

System and method embodiments allow ablative energy to be delivered to an ablation device (such as a catheter) in a controlled manner while preventing popping of tissue due to expansion of bubbles that are introduced into or formed within tissue when the tissue is heated to a sufficiently high temperature, e.g., when tissue is overheated during a tissue ablation procedure. Embodiments are operable by detecting ultrasonic energy that is emitted by bubbles as the bubbles resonate or collapse in tissue. Thus, embodiments are operable in a manner that is in contrast to known systems and methods that rely on expansion and popping of tissue due to bubble expansion to determine that tissue popping by bubble expansion has occurred and that the level of ablation energy should then be reduced.

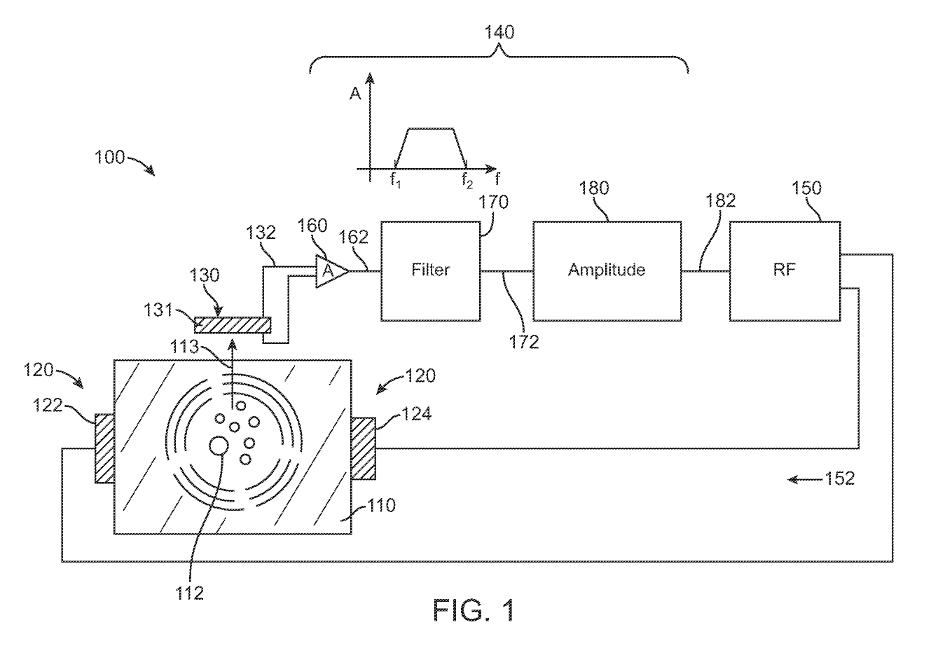

Thus, embodiments are capable of achieving effective tissue ablation without tissue popping caused by bubble expansion, thereby eliminating the negative effects associated with such tissue popping including tearing of tissue and release of solid emboli or micro-bubbles into the blood, which may interrupt blood flow and lead to distal circulatory damage or stroke. Further, embodiments are capable of eliminating “popping” sounds that are associated with known systems and techniques, thereby making ablation procedures more comfortable and less stressful for patients and less worrisome for clinicians that administer ablative energy to patients. Further aspects and advantages of embodiments are described with reference to FIGS. 1-8B.

Referring to FIG. 1, a system 100 constructed according to one embodiment for controllably delivering ablation energy to tissue 110 to prevent tissue popping by bubble expansion includes an ablation device 120, a detector 130, a control element 140 and a source 150 of ablation energy or current 152. Ablation energy 152 is provided to tissue 110, thereby heating the tissue 110. Bubbles 112 are generated within the tissue 110 if the tissue is overheated.

In the illustrated embodiment, the detector 130 is configured to detect ultrasonic energy 113 that is spontaneously emitted by bubbles 112, e.g., bubbles 112 that pulsate, shrink or collapse (i.e., bubbles that do not pop tissue due to bubble expansion) and that resonate within tissue 110. The detector output 132 is provided to the control element 140, which adjusts the energy source 150 and the amount of ablation energy or current 152 that is provided to the ablation device 120 such that bubbles 112 do not expand and explode.

In the illustrated embodiment, the energy source 150 is a radio frequency (RF) energy source or RF generator. It should be understood, however, that other energy sources besides a RF generator 150 may be utilized such as a high intensity focused ultrasonic energy source. For ease of explanation, reference is made to a RF energy source or RF generator 150 that generates ablation energy or electrical current 152 that is provided to an ablation device 120. In the illustrated embodiment, the ablation device 120 is generally illustrated as including a RF electrode 122 and a return electrode 124 for conduction of energy to and from the tissue 110.

In the illustrated embodiment, the detector 130 is a single ultrasound transducer or receiving transducer 131 (generally referred to as receiving transducer 131). According to one embodiment, the receiving transducer 131 includes known piezoelectric members and may be formed from various known ceramic and crystalline materials, e.g., various species of lead-zirconate-titanate (PZT) ceramics including, but not limited to, PZT-5H, PZT-5A, PZT-4, and PZT-8. The receiving transducer 131 may also be made of a plastic material such as polyvinyladine film and other suitable materials as appropriate.

In the illustrated embodiment, the receiving transducer 131 is configured to detect ultrasonic energy 113 that is spontaneously emitted by collapsing or pulsating bubbles 112 (as opposed to expanding and exploding tissue popping bubbles) that resonate within tissue 110. The output 132, e.g., a voltage signal, generated by the receiving transducer 131 is provided to the control element 140.

In the illustrated embodiment, the control element 140 includes one or more amplifiers 160 (one amplifier is illustrated for ease of illustration), one or more filters 170 (one filter is illustrated for ease of illustration) and an amplitude measuring element 180.

Although certain components described in this specification are described as being part of the control element 140, it should be understood that such components may be separate from the control element 140, and that the control element 140 may include other components than the components illustrated in FIG. 1, as shown in other Figures. For example, the amplifier 160 may be a component of the control element 140 or a separate component. Accordingly, Figures showing control element 140 components are provided for purpose of illustration and as examples of how embodiments may be implemented. For ease of explanation, reference is made to a control element 140 that includes components connected between the receiving transducer element 131 and the RF generator 150, although embodiments are not so limited.

The voltage signal 132 generated by the receiving transducer 131 is provided as an input to the amplifier 160. The amplifier 160 amplifies the voltage signal 132, and the amplified signal 162 is filtered 170. One example of a filter 170 that may be utilized for this purpose is a band-pass filter, as shown in FIG. 1. In the illustrated embodiment, the band-pass filter 170 is configured to pass signals or energy at frequencies between a first frequency f₁ and a second frequency f₂, wherein f₁<f₂. The filter 170 is operable to filter out frequencies outside of this range. In one embodiment, the frequency f₁ may be above the auditory range (e.g., greater than about 20 kHz), and the frequency f₂ may be less than or equal to about 10 MHz. Other frequencies and frequency ranges may be utilized as appropriate, and frequencies of 20 kHz and 10 MHz are provided as examples of how embodiments may be implemented.

The output of the filter 170, or the filtered signal 172, is provided as an input to the amplitude measurement element 180 (hereafter referred to as amplitude element 180). The output 182, or amplitude measurement or data, provided by the amplitude element 180 is provided as an input to the RF generator 150 and serves as a control or feedback parameter. The RF generator 150 includes logic or other suitable control components, hardware and/or software that may be adjusted or configured based on the received amplitude data 182 in order to adjust and control the RF ablation current 152 that is output by the RF generator 150 and provided to tissue 110. In this manner, the sizes or dimensions and number of bubbles 112 remain sufficiently small, and tissue 110 popping that would be caused by expansion of bubbles is advantageously prevented or substantially reduced with embodiments.

More specifically, bubbles 112 initially form within the tissue 110 as a result of super-saturation that is caused by overheating of tissue. Whether bubbles 112 form, and the size and number of bubbles 112 that form, may depend on various factors including, for example, tissue 110 temperature, movement of an ablation probe and blood flow, which may cool heated tissue. Bubbles 112 may shrink in size or pulsate due various factors including, for example, higher pressures, lower temperatures, condensation of gas, and the bubbles 112 dissolving in water. When bubbles 112 shrink in size, for example, the resonating bubbles 112 emit ultrasonic waves at a resonant frequency fₚ and its sub-harmonics and harmonics. The resonant frequency is proportional to the inverse of the bubble 112 diameter. For example, the relationship between a size of a bubble 112 and the resonant frequency of a bubble 112 has been expressed as r_{d}=3.28/fₚ where r_{d} is a radius of the bubble 112 in micrometers, and fₚ is the resonant frequency in MHz.

The amplitude of the control or feedback output 182 increases with the size and number of bubbles 112 that are present, thereby ensuring that the RF generator 150 is controlled in such a manner that bubbles 112 remain sufficiently small in number and size, do not expand and explode and do not result in tissue popping. More particularly, high frequency ultrasonic energy is emitted by bubbles 112 at a frequency related to the diameter of the bubble 112 (as discussed above), and a bubble 112 can be considered to be a high Q resonator such that the resonance is relatively sharp in frequency. For example, embodiments may be utilized to detect high frequency ultrasonic energy emitted by bubbles 112 having diameters of about 1 micrometer to about 100 micrometers, e.g., about 10 micrometers. Such “micro” bubbles 112 do not pop due to expansion and, therefore, do not result in tissue popping by bubble expansion. Collapsing bubbles 112 having a diameter of about 10 micrometers, for example, emit ultrasonic energy having a resonant frequency of about 150 kHz, which rises to a frequency that is higher than 1 MHz as the bubble 112 completely collapses.

With embodiments, such high frequency ultrasound detection can be distinguished from lower frequency sounds, e.g., the sound of a beating heart, a human voice and other environment sounds. Since the bubbles 112 present in the tissue 110 due to overheating have a random size distribution, the ultrasonic energy emitted by bubbles 112 collectively adds to form broad band sound, which is related to the aggregate size and number of the bubbles 112, and not the popping of the tissue 110 due to expansion of bubbles 112.

Embodiments, therefore, are able to prevent tissue popping caused by bubble expansion by utilizing a control or feedback signal or circuit 140 that adjusts the output 152 of the RF generator 150 based on a desired small number and small dimensions of bubbles 112 rather than other parameters (e.g., temperature or adjusting energy after tissue popping by bubble expansion has occurred or been audibly detected). Accordingly, embodiments function in substantially different manner than certain known systems that detect sounds generated by tissue that actually pops due to bubble expansion and explosion. In this regard, embodiments function in a manner that is the opposite of certain known systems.

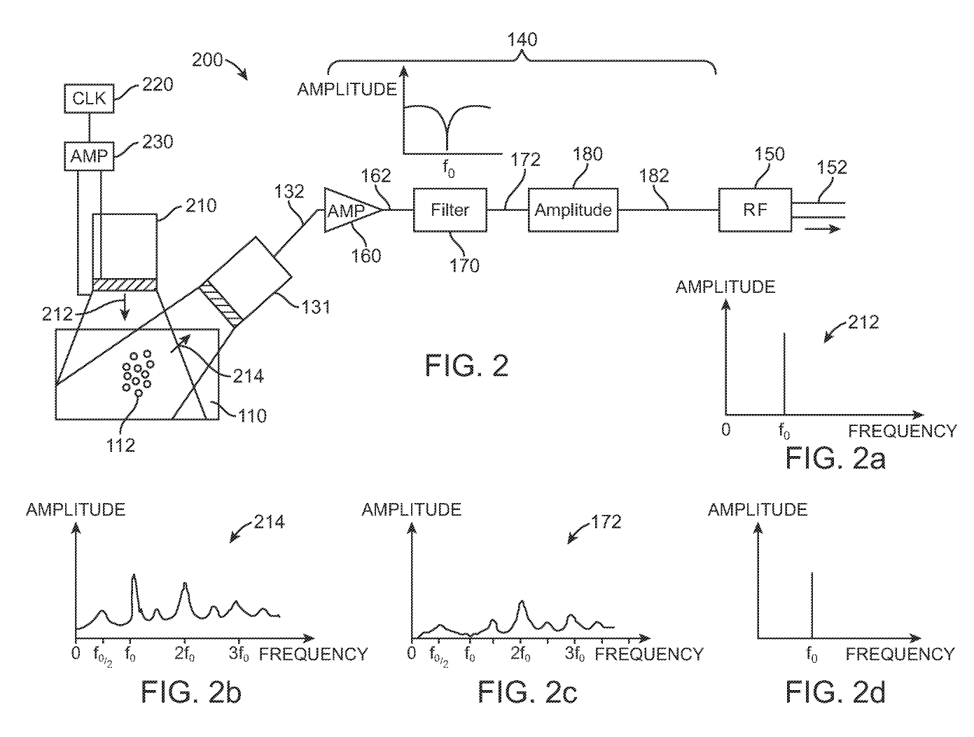

Referring to FIG. 2, a system 200 constructed according to another embodiment and configured to controllably deliver energy to an ablation device to prevent tissue popping resulting from bubble expansion includes certain components described above with reference to the system 100 shown in FIG. 1. For ease of reference, common reference numbers are used to identify the same or similar components and the manner in which these components function is not repeated.

In the illustrated embodiment, the system 200 includes an additional ultrasound transducer element 210 compared to the embodiment shown in FIG. 1. For ease of explanation, the ablation device 120, e.g., a catheter, is omitted from FIGS. 1-6, but illustrated in other Figures. In the illustrated embodiment, the system 200 includes an emitting transducer 210 and a receiving transducer 131 (e.g., as generally described above with reference to FIG. 1). However, rather than detecting spontaneous emission of ultrasonic energy from bubbles 112 using a single receiving transducer 131, the system 200 is configured to utilize interrogation signals 212 and emission signals 214 in order to obtain data related to the number and dimensions of bubbles 212 for purposes of adjusting the RF generator 150 to maintain small bubble 112 sizes and to prevent tissue popping by bubble expansion.

The system 200 also includes a clock 220 or other suitable component for generating an insonation or interrogation signal 212, and one more additional amplifiers 230 (one amplifier is shown) as needed. In certain instances, the interrogation signal 212 may be a clock signal, e.g., a square wave or a sine wave. For ease of explanation, reference is made to an interrogation signal 212 generally, although certain figures may illustrate clock components 220 and an amplifier 230 as needed for generating an interrogation signal 212.

During use, the clock 220 is used to drive the emitting transducer 210 to emit an interrogation signal 212 at frequency f₀ (as shown in FIG. 2A). The interrogation signal 212 insonates the tissue 110 with ultrasonic energy Ultrasonic energy in the interrogation signal 212 reflects from interfaces where the acoustic impedance changes, such as at tissue 110 boundaries. In addition to having ultrasonic energy reflected at the drive or insonation frequency f₀, bubbles 112 also emit ultrasonic emit energy at various harmonics and sub-harmonics of the insonation frequency f₀ as shown in FIG. 2B. The resulting emission signal 214 emitted by bubbles 112 at one or more different frequencies is detected by the receiving transducer 131. For this purpose, receiving transducer 131 may be very wide band in its sensitivity.

More specifically, when small or micro bubbles 112 in tissue 110 are insonated by the signal 212, the bubbles 112 resonate, similar to the ringing of a bell. This bubble 112 resonance is a nonlinear phenomenon. In response to insonation by ultrasonic energy 212 at a single frequency f₀, bubbles 112 close to a resonant size will resonate and emit ultrasonic energy not only at the drive or interrogation frequency f₀, but also at sub-harmonic and harmonic frequencies n×(f₀/2) wherein n=1, 2, 3, etc. (as shown in FIG. 2B). The emission signal 214 is detected by the receiving transducer 131, which generates a corresponding output 132 that is provided as an input to an amplifier 160, the output 162 of which is filtered 170.

In the illustrated embodiment, the filter 170 is a band reject or notch filter that removes or filters the insonation frequency f₀ from the emission signal 214 in order to generate a modified signal or filtered output 172. The filtered output 172 includes sub-harmonics and harmonics of f₀, but not f₀ itself (as shown in FIG. 2C). Embodiments that utilize sub-harmonics and harmonics in this manner provide a number of benefits and advantages. For example, an interface having a change of acoustic impedance generates reflections, which are the basis of ultrasound images. However, bubbles 112 generate substantial levels of sub-harmonics and harmonics. Thus, while it may be difficult to distinguish bubbles 112 from tissue by reflection, embodiments provide the ability to determine whether or not there are bubbles 112 present by measuring the level of non-linear emissions by analyzing harmonics and sub-harmonics.

The resulting filtered or “notched out” signal 172 is provided as an input to the amplitude element 180, which measures the amplitude of the signal 172, e.g., using a true root mean square (RMS) converter or other suitable components and techniques. The resulting output 182 is provided as an input to the RF generator 150 as a control or feedback parameter. The RF generator 150 generates RF ablation current 152 for performing RF ablation on the tissue 110, as controlled and adjusted by the input 182. In this manner, the number and sizes of bubbles 112 remain sufficiently small to prevent tissue popping by bubble expansion. For example, the RF generator 150 may increase the RF ablation power, up to a user setting, while the stimulated emission is below a threshold amplitude, and limit or decrease the RF ablation power to keep emissions below the desired threshold and maintain small bubble 112 dimensions.

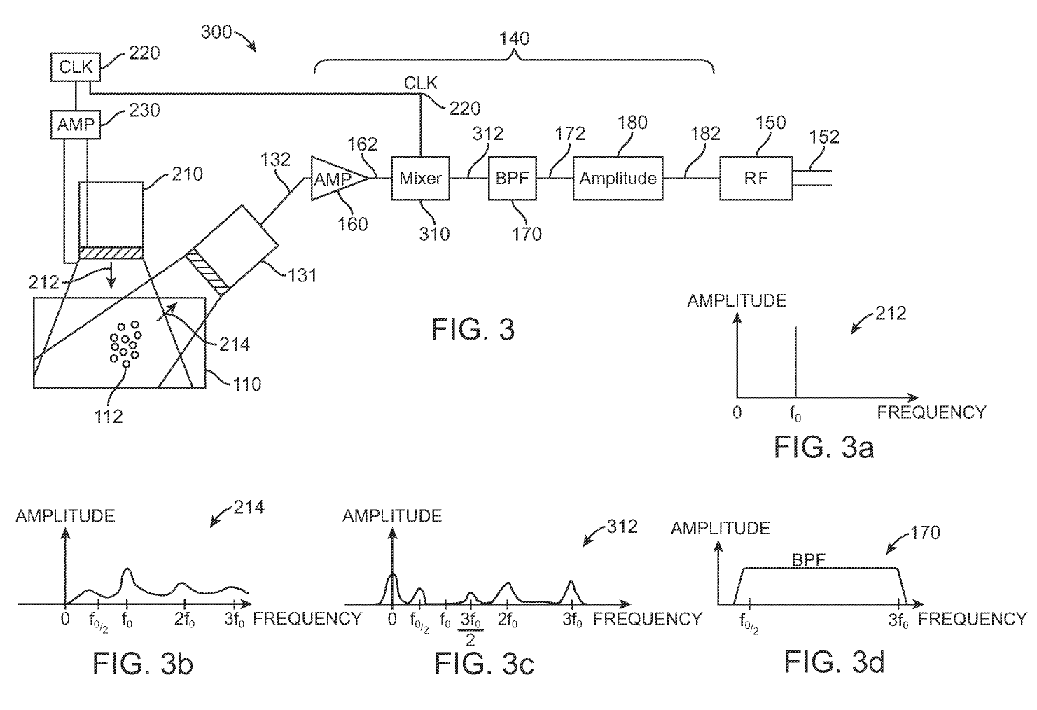

Referring to FIG. 3, a system 300 constructed according to another embodiment and configured to controllably deliver energy to an ablation device 120 to prevent tissue popping by bubble expansion includes certain components described above with reference to the systems 100 and 200 shown in FIGS. 1-2. The system 300 also includes different control element 140 components that may be used instead of a notch filter 170 to remove the interrogation frequency component f₀ from the emission signal 214 emitted by bubbles 212.

More particularly, in the illustrated embodiment, the emission signal 214 (one example of which is shown in FIG. 3B), is detected by the receiving transducer 131, amplified 160, and provided as an input to a mixer 310. The mixer 310 multiplies the amplified signal 162 and the interrogation signal 212 (one example of which is shown FIG. 3A), to obtain a resulting mixed signal or output 312 (one example of which is shown in FIG. 3C). As shown in FIG. 3C, mixing the interrogation signal 212 and the amplified output 162 effectively removes the interrogation frequency f₀ from the amplified signal 162 to produce a mixed signal 312 that includes sub-harmonics (f₀/2) as well as harmonics (n×f₀ for n=1, 2, 3, etc.) of the interrogation frequency f₀.

In another embodiment, the mixer 310 may be provided with a frequency of f₀/2 rather than f₀ as a homodyne receiver to allow narrowband detection near f₀/2. In this embodiment, the output 312 of the mixer 310 may be provided through a low pass filter rather than a band pass filter. In a further embodiment, the mixer 310 may be provided with a frequency of 2f₀ for detection near 2f₀. It should be understood that different frequencies and system components may be utilized as necessary.

The mixed signal 312 is provided as an input to a filter 170 which, in one embodiment as illustrated, is a band-pass filter. An example of one manner in which the band-pass filter 170 may function is shown in FIG. 3D). In the illustrated embodiment, the filter 170 includes a band-pass filter spectrum from the sub-harmonic f₀/2 to the harmonic 3f₀ and generates an output signal 172. The output signal 172 retains frequencies from approximately f₀/2 to 3f₀. The amplitude of the resulting band-pass filtered signal 172 is measured by the amplitude element 180. The output 182 of the amplitude element 180, which is monotonically related to the peak voltage, or the average power of the signal, is used to adjust or control the RF generator 150, as described above with reference to other embodiments.

In alternative embodiments, interrogation signals 212 at multiple different frequencies may be utilized rather than an interrogation signal 212 at a single interrogation frequency f₀. Using multiple interrogation 212 frequencies provides an advantage of interrogating bubbles 112 having a wider range of diameters.

For example, an interrogation frequency range of approximately 20 kHz to 1 MHz may be utilized to interrogate bubbles 112 having diameters of about 164 μm to about 3 μm, or about a 50:1 ratio. More specifically, as discussed above, one manner in which the resonant frequency of a bubble 112 may be expressed relative to frequency is r_{d}=3.28/fₚ, where r_{d} is a radius of a bubble 112 in micrometers, and fₚ is a resonant frequency a bubble 112 in MHz. In embodiments, the frequency f₀ of the interrogation signal 212 may be about 20 kHz for interrogating bubbles 112 having a diameter of about 165 micrometers. The interrogation frequency f₀ may be about 200 kHz for interrogating bubbles 112 having a diameter of about 16.5 micrometers. Bubbles 112 having a diameter of about 16.5 micrometers also have a sub-harmonics resonant frequency at about 100 kHz. The frequency f₀ of the interrogation signal 112 may also be about 2 MHz for interrogating bubbles 112 having a diameter of about 1.65 micrometers.

In addition to detecting the amplitude of the primary emission 214 at the interrogation signal 112 frequency f₀, detection of the other emissions 214 offers significant benefits for the control of RF ablation current 152. One such benefit is the lack of interfering signals. For example, the frequency of the emission energy 214 or interrogation signal 212 may be sufficiently high, e.g., about 40 kHz, such that the emission signal 214 is easily filtered to remove physiological sounds since the sub-harmonic emission will be at about 20 kHz. Examples of such sounds include a heart beating, respiration, gastric motion, vocalizations by the patient and other sounds in the environment. This provides the significant benefit of lack of interfering signals.

It should be appreciated that embodiments may be implemented with other system configurations. Further, signal processing functions may be satisfied with a spectrum analyzer (not illustrated). With this system configuration, an operator may visually observe f₀/2 or 2f₀ and manually adjust the radio frequency ablation to avoid generation of bubbles 112.

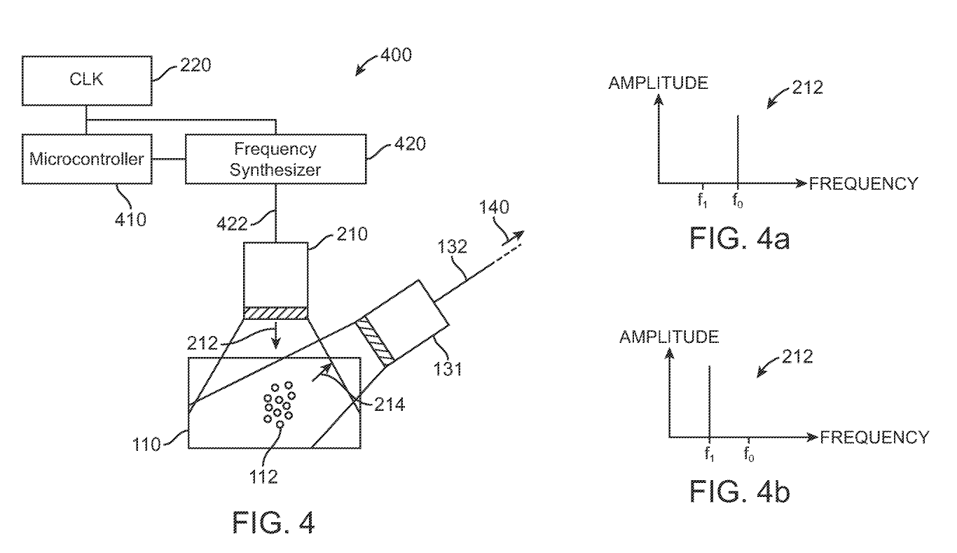

According to one embodiment, an ultrasound interrogation signal 212 may be a band limited spread spectrum signal. There are several techniques for producing a band limited spread spectrum signal. One technique involves use of a frequency generator (such as an oscillator). The frequency generator produces a signal that jumps between two or more different frequencies, otherwise referred to as “frequency hopping.” With this technique, as described in further detail with reference to FIG. 4, the resulting interrogation signal 212 comprises multiple frequencies, but at any given time, the interrogation signal 212 comprises one discrete frequency. In this manner, “frequency hopping” embodiments are stepwise versions of a swept frequency clock 220. A smooth swept frequency clock may likewise be utilized. Bubbles 112 are resonant at a frequency related to their size. Thus, while certain embodiments may be successfully utilized to measure bubbles 112 having a narrow range of sizes using a fixed frequency (e.g., as shown in FIG. 2), frequency hopping embodiments may be used to sweep multiple frequencies to detect bubbles 112 of various sizes or various ranges of sizes.

Referring to FIG. 4, a system 400 constructed according to one embodiment that utilizes “frequency hopping” for controlling RF ablation current 152 provided to an ablation device 120 to prevent tissue popping by bubble expansion employs sub-harmonic emission control interrogates tissue 110 with a band limited frequency hopping signal, excluding the band of frequencies from the received emission signal 214, and using amplitude output 182 as a control parameter to control the RF generator 150. In the illustrated embodiment, a microcontroller 410 is operably coupled to a frequency synthesizer integrated circuit 420 (generally referred to as synthesizer 420). The microcontroller 410 causes the synthesizer 420 to generate a drive signal 422 at a desired frequency. The microcontroller 410 and the synthesizer 420 may share a common clock 220 (as illustrated) or they may have separate clocks. The synthesizer 420 generates the drive signal 422 after receiving control instructions from the microcontroller 410, and the drive signal 422 is provided to the transducer element 210, which emits an interrogation signal 212 at a first frequency f₀ (as shown in FIG. 4A). After a period of time, the microcontroller 410 instructs the synthesizer 420 to produce a drive signal 422 at a different frequency f₁, (as shown in FIG. 4B), and so forth, resulting in interrogation signal 212 having two or more discrete frequencies (f₀, f₁, . . . fₙ). According to one embodiment, the discrete frequencies (f₀, f₁, . . . fₙ) are not integer multiples of each other. In this manner, sub-harmonic frequencies of one discrete frequency may be separated or distinguished from sub-harmonic frequencies of another discrete frequency. After each frequency hop, the output may be blanked for a brief time to allow the return from the previous transmission to cease.

This results in the synthesizer 420 generating a band limited spread spectrum drive signal 422, which is used to drive the transducer 210 to emit an interrogation signal 212 that includes harmonics and sub-harmonics based on the drive signal 422. As a result, bubbles 112 that are present within the tissue 110 emit energy or a signal 214 that is detected by the receiving transducer 131. The signal or voltage output 132 generated by the receiving transducer 131 is then processed as described above in other embodiments, to control the current 152 generated by the RF generator 150 and ensure that the number of dimensions of bubbles 112 remain sufficiently small to prevent tissue popping by bubble 112 expansion.

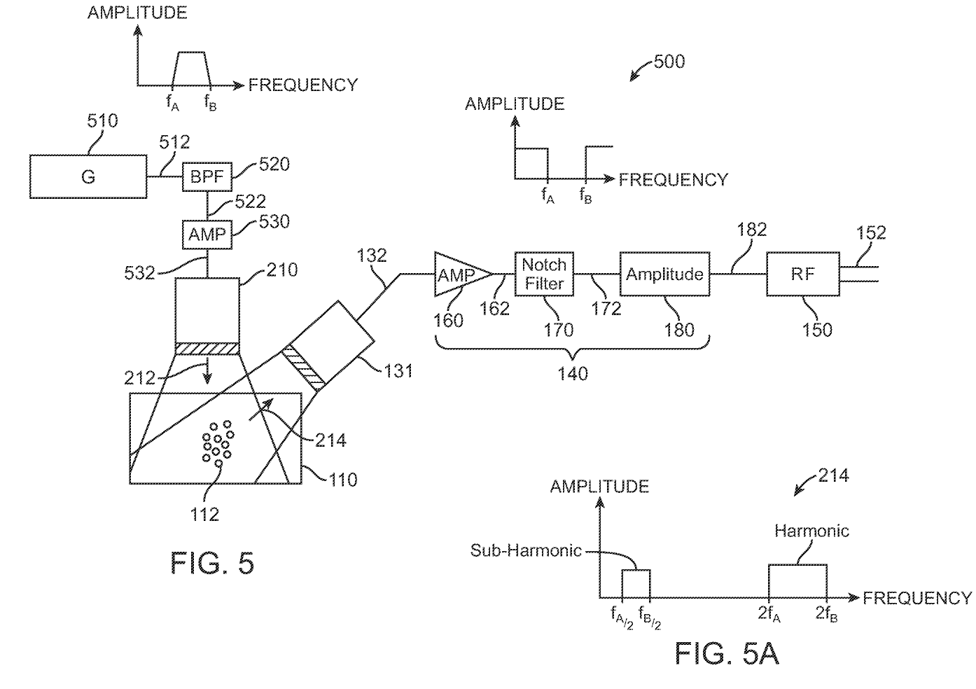

Referring to FIG. 5, a system 500 constructed according to another embodiment employs band limited white noise sub-harmonic control. The system 500 produces a band limited spread spectrum signal utilizing a spread spectrum generator, such as a white noise generator 510. The output 512 of the white noise generator 510 is provided as an input to a band-pass filter (BPF) 520 such that the output 512 is filtered from a first frequency f_{A} to a second frequency f_{B} using the filter 520. In one embodiment, f_{B}/F_{A}<2. The resulting band-pass filtered signal 522 is provided as an input to an amplifier 530, the amplified output 532 of which drives the ultrasound transducer 210, which may be a broadband ultrasound transducer, which emits an interrogation signal 212 and insonates tissue 110 and interrogates bubbles 112. If there are any bubbles 112 whose sizes lie within the range corresponding to the band-pass frequency range f_{A} to f_{B}, these bubbles 112 will resonate and emit energy 214 having sub-harmonics and harmonics and that is received by receiving transducer 131. FIG. 5A illustrates such a spectrum of the emission signal 214 including sub-harmonics from f_{A}/2 to f_{B}/2, emission energy having frequencies from f_{A} to f_{B}, and harmonics from n×f_{A} to n×f_{B}, for n=2, 3, 4, etc.

The resulting output or voltage signal 132 generated by the broadband receiving transducer 131 is amplified 160 to generate an amplified output 162. A filter 170, such as a band reject filter, is used to remove the frequency range f_{A} to f_{B}, thereby producing a filtered or notched out signal 172 that includes sub-harmonics and harmonics of the amplified emission signal 162, but not emission energy at frequencies of f_{A} to f_{B}. In another embodiment, the filter 170 may be omitted and broadband emissions by the bubbles 112 may be measured. The signal 172 output by the filter 170 is provided to the amplitude element 180, which measures the amplitude of signal 172 and generates a corresponding output 182 to control the RF generator 150, as described above relative to other embodiments.

In some cases, it may be beneficial to utilize transducers 131, 210 that are made of plastic rather than other materials (such as ceramic) in embodiments that utilize multiple frequencies and spread spectrums. Plastic transducer materials such as polyvinyladine fluoride film may be particularly suited for such applications due to the resulting transducers 131, 210 being thin, having high resonant frequency (since frequency related to thickness), wide bandwidth for transmission and receiving of signals, and a wide sensitivity range when used below their resonant frequency.

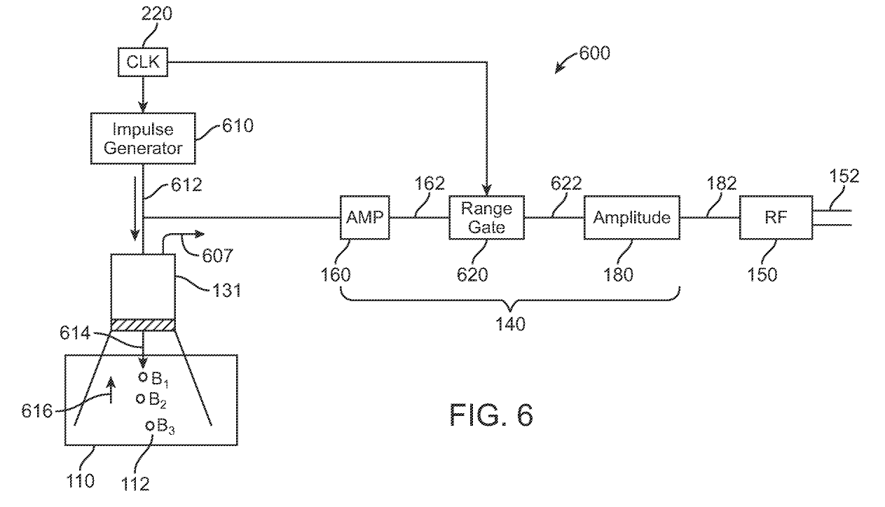

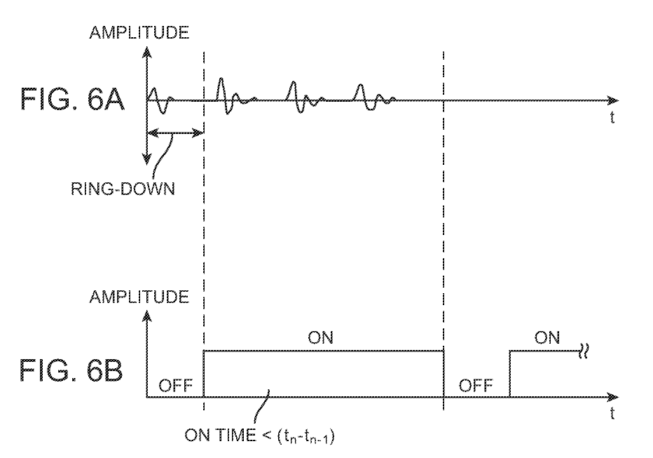

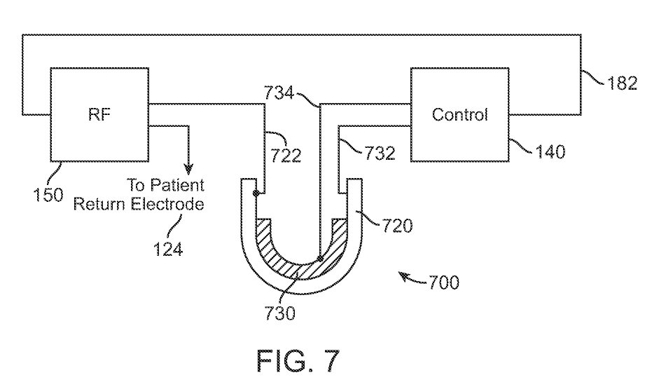

Further, referring to FIG. 6, a system 600 constructed according to another embodiment for controllably delivering ablation energy to an ablation device while preventing tissue popping by bubble expansion utilizes a short time duration wide-band impulse to first interrogate bubbles 112, and then stops the interrogation signal and listens with the transducer 131 for any resonance of the bubbles 112. In such an embodiment, the bubbles 112 effectively become resonant circuits that may be excited by an interrogation impulse and respond according to their harmonics and sub-harmonics, and their emission 214 includes energy at different harmonics and sub-harmonic frequencies.

More particularly, in the illustrated embodiment, an impulse generator 610 generates an impulse 612 at time to. This impulse 612 serves to drive the transducer 131. In response, the transducer 131 generates an interrogation impulse 614 that is applied to tissue 110. When the impulse 614 encounters a bubble 112 in tissue 110, the bubble 112 resonates and emits a signal 616 that can be detected sensed by the same ultrasound transducer 131. In response, the transducer 131 generates an output or sensed signal 607, which is amplified 160. The amplified output 162 is provided to a range gate 620, which serves as an ON/OFF switch for the amplified signal 162 as shown in FIG. 6A, which illustrates an example set of three representative bubbles 112 (B₁, B₂, B₃).

As shown in FIG. 6A, the impulse 612 is generated at time to, and bubbles 112 B₁, B₂ and B₃ emit ultrasonic energy or emissions 616 that are represented at increasing times t₁, t₂ and t₃, respectively, due to the fact that bubble B₁ is closer to transducer 131 than bubble B₂, which in turn is closer than bubble B₃. The range gate 620 is in an OFF state at time t₀ and switched to an ON state after a time interval (“ringdown period”), as shown in FIGS. 6A-B. Bubble emissions 616 that occur after the range gate 620 switches to an ON state are reflected in range gate outputs 622. The amplitudes of the range gate outputs 622 are measured by the amplitude element 180, and the resulting output signal 182 controls or adjusts the RF generator 150, as described in previous embodiments. By tuning the timing of the ON and OFF states of the range gate 620, a distance bracket (as measured from transducer 602) then can be selected within which bubbles 112 are detected, and the amplitude or power of the signal 182 is proportional to the size and number of bubbles 112 present in tissue 110.

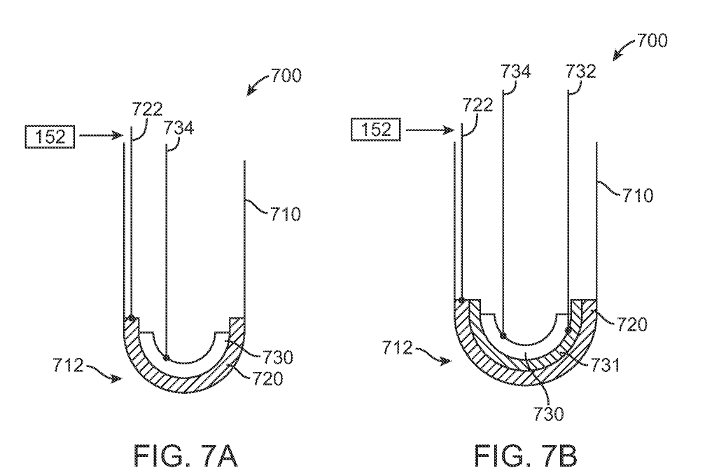

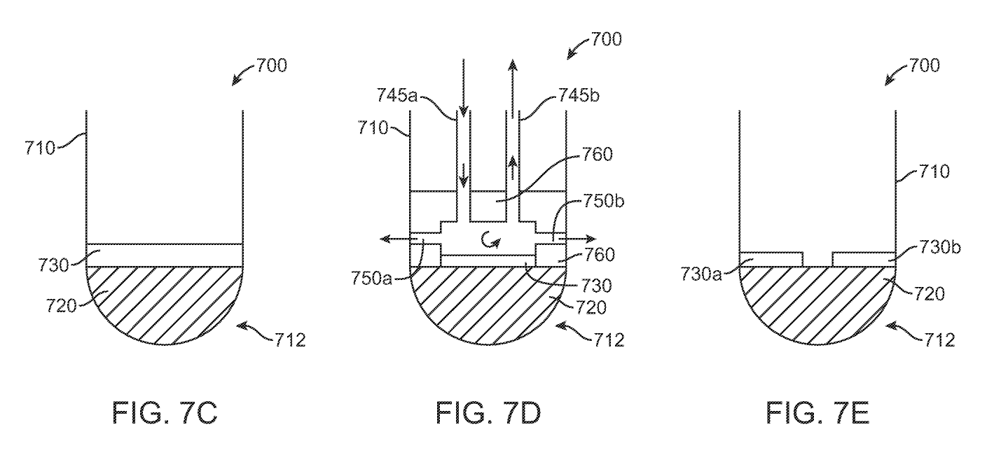

FIGS. 7 and 7A-B illustrate other manners in which embodiments may be incorporated within an ablation catheter 700 for controllably delivering ablative energy 152 to the catheter 700 while preventing tissue popping caused by expansion of bubbles 112. With reference to FIGS. 7 and 7A, the ablation catheter 700 includes an elongate body 710 (e.g., a plastic body), a metal ablation tip 720 (e.g., a RF electrode) and an ultrasound transducer 730 positioned at a distal end 712 thereof. In the illustrated embodiment, a wire or connection 722 is provided for delivering RF energy to the ablation tip 720, a wire or connection 734 connects to a back side of the ultrasonic transducer 730, and a wire or connection 732 connects to a front side of the transducer 730. FIG. 7B illustrates similar components and an insulation element 731 that is disposed between the transducer 730 and the electrode 720. The insulation element 731 may be used to isolate the transducer 730 from the electrode 720, e.g., to isolate the transducer 730 from noise generated by high voltage signals used for RF ablation. In the configurations shown in FIGS. 7 and 7A-B, the transducer 730 is operably coupled to embodiments of control elements 140 as described above with reference to FIGS. 1-6 to transmit interrogation signals and receive emission signals 212, 214, and thereby generate a control signal (e.g., output 182) to control the RF generator 150 and maintain sufficiently small bubble 112 dimensions to prevent tissue popping due to bubble expansion.

FIGS. 7C-E illustrate ablation catheters 700 constructed according to other embodiments. FIG. 7C illustrates an ablation catheter 700 having a solid ablation tip 720 at a distal end 712 of the elongate body 710 and a flat disc-like shaped ultrasound transducer 730 adjacent to the ablation tip 720. FIG. 7D illustrates an ablation catheter 700 that is irrigated and may incorporate embodiments. In the illustrated embodiment, the body 710 of the catheter 700 has one or more inlet and outlet tubes 745a, 745b and defines one or more outlets or apertures 750a, 750b through which a coolant fluid may flow (as generally illustrated by circulation arrow), and heat transfer may be accomplished by one or more heat exchange elements 760. FIG. 7E illustrates another ablation catheter 700 that may incorporate embodiments and that includes a solid ablation tip 712 and flat half-disc-shaped ultrasound transducers 730a, 730b. One transducer 730a is for transmitting an interrogation signal 212, and the other transducer 730b is for receiving an emission signal 214.

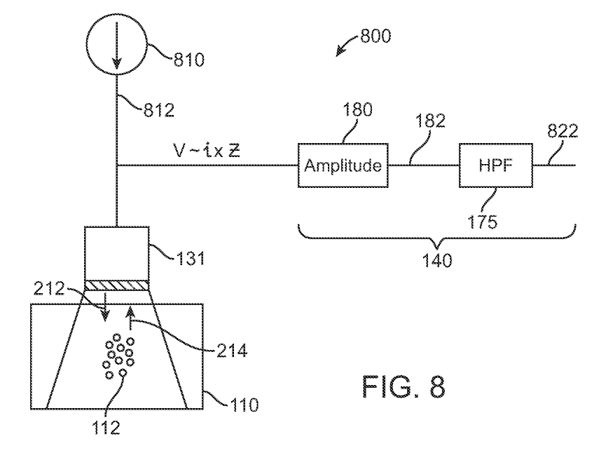

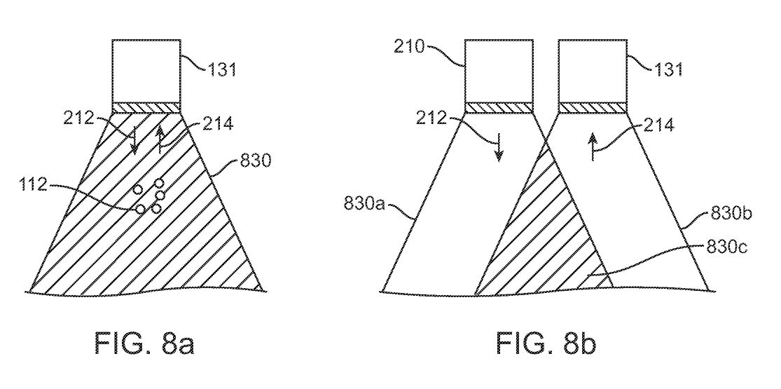

Referring to FIG. 8, a single crystal continuous wave Doppler system 800 constructed according to a further alternative embodiment includes a single transducer element 131 that is used for emitting continuous wave or pulsed interrogation signals 212 and detecting continuous emitted energy or signals 214 (whereas in the embodiment shown in FIG. 1, a single transducer element 131 is utilized to detect spontaneous ultrasonic energy 113 emitted by bubbles 112 without interrogation or insonation). In the illustrated embodiment, the system 800 includes a RF constant current source 810 that generates a constant current 812 at some frequency f₀. The constant current 812 is provided into the single transducer 131, which insonates tissue 110 with an ultrasound interrogation signal 212 at a frequency f₀. An amplitude element 810 measures the amplitude of the voltage across the transducer 131. This voltage V is approximately i×Z, wherein i is the constant current 812 generated by the RF constant current source 810, and Z is the impedance of the transducer 131 at a frequency f₀.

As the interrogation signal 212 bounces off of the moving surface of contracting bubbles 112 in the tissue 110, the interrogation signal 212 is Doppler-shifted. The resulting shifted emission signal 214 is detected by the same transducer 131, and the amount of the Doppler-shift adds linearly to the voltage V across the transducer 131. The Doppler-shifted interrogation signal 212 has a frequency that is proportional to the rate of collapse of a bubble 112, and the amplitude of the interrogation signal 212 reflection, which is proportional to the size and number of bubbles 112. Therefore, the output 182 of the amplitude element 180 is the sum of the constant direct current (DC) voltage as given by V=i×Z and the Doppler-shift representing the detected amplitude of ultrasound frequency shifted signal 214.

The output 182 of the amplitude element 180 is provided as an input to, for example, a high pass filter 175. The voltage level at the output 182 may be large due to the constant RF current and the constant impedance of the tissue and transducer. A small AC audio frequency Doppler signal may also be a component of the output 182. A high pass filter 175 or other suitable component or filter may be used to remove large DC components of the output 182, resulting in an audio signal. The output 822 of the filter 175 may then be used in the above embodiments just as if it had been generated by a second receiving transducer 210 (e.g., as shown in FIG. 2) described above in various other embodiments. Embodiments may utilize various Doppler shifting technique and components. One manner in which embodiments may utilize Doppler-shifting techniques and processing resulting data is described in V. L. Newhouse, et al. in “Bubble size measurements using the nonlinear mixing of two frequencies.” J. Acoust. Soc. Am. 75(5), May 1984, the contents of which are incorporated herein by reference as though set forth in full.

FIGS. 8A-B illustrate advantages that may be achieved when using a single transducer 131 and associated system 800 components to both insonate tissue 110 and detect emission signals 214. Referring to FIG. 8A, utilizing a single transducer 131 to emit an interrogation signal 212 and receive or detect a emission signal 214 utilizes the entire beam 830 of the transducer 131. Referring to FIG. 8B, separate insonation and detection transducers 131, 210 may limit the sensitive area to the overlapping portion 830c of the transmit beam 830a and the receive beams 830b of respective transducers 210, 131. Additionally, use of a single transducer 131 for insonation and detection eliminates the need for coordinating the orientation of the separate interrogation and detection transducers 210, 131 so that the beam overlap 830c coincides with the portion of the tissue 110 where detection of bubbles 112 is desired. This is particularly advantageous for small transducers that are utilized with small diameter catheters. Thus, different embodiments have different advantages, and system configurations may be selected based on, for example, available system components and detection capabilities.

Although particular embodiments have been shown and described, it should be understood that the above discussion is not intended to limit the scope of these embodiments. Various changes and modifications may be made without departing from the scope of the claims.

For example, embodiments may be configured to include a single transducer element or multiple transducer elements. Moreover, embodiments may be configured to detect spontaneous ultrasonic energy without a separate interrogation signal or may insonate tissue with an interrogation signal and detect a resulting emission signal. Moreover, embodiments may be implemented in various types of ablation devices, one example of which is a catheter. Further, it should be understood that embodiments may be implemented to prevent tissue popping resulting from bubble expansion by maintaining bubble sizes or diameters within different ranges so long as tissue popping caused by expansion and popping of bubbles does not occur.

Thus, embodiments are intended to cover alternatives, modifications, and equivalents that may fall within the scope of the claims.

What is claimed is:

1. A system for controllably delivering ablation energy to tissue, comprising:
an energy transmitter operable to transmit ablation energy into body tissue;
an ultrasound detector configured to detect energy emitted by collapsing or shrinking bubbles resonating in body tissue receiving ablation energy transmitted by the energy transmitter; and
a control element operatively coupled to the energy transmitter and ultrasound detector, the control element configured to adjust an amount of ablation energy being transmitted by the energy transmitter in response to the energy detected by the ultrasound detector.

2. The system of claim 1, wherein the detector is configured to detect energy at a resonant frequency based on a particular bubble size.

3. The system of claim 2, wherein the control element is configured to adjust the ablation energy to maintain bubble diameters at less than about 100 micrometers.

4. The system of claim 1, wherein the control element is configured to adjust the ablation energy based on an amplitude of the detected energy.

5. The system of claim 1, wherein the detector is configured to detect energy emitted by collapsing bubbles at a frequency higher than a frequency of a beating heart sound wave.

6. The system of claim 1, the ultrasound detector comprising a first transducer element, the system further comprising a second transducer element configured to insonate body tissue receiving ablation energy from the energy transmitter with an interrogation signal.

7. The system of claim 6, the interrogation signal comprising a single interrogation frequency.

8. The system of claim 6, the interrogation signal comprising a band limited spread spectrum signal.

9. The system of claim 6, wherein the interrogation signal is transmitted at a first frequency, and the energy emitted by collapsing or shrinking bubbles has a second frequency.

10. The system of claim 9, wherein the second frequency comprises a plurality of harmonics or sub-harmonics of the first frequency.

11. The system of claim 9, the second frequency being a harmonic or a sub-harmonic of the first frequency.

12. A method of controllably ablating body tissue, comprising:
applying ablation energy to body tissue;
detecting ultrasound energy emitted by collapsing or shrinking bubbles that resonating within the body tissue receiving the ablation energy; and
adjusting the ablation energy being applied to the body tissue in response to the detected energy.

13. The method of claim 12, wherein detecting ultrasound energy by collapsing or shrinking bubbles comprises detecting ultrasound energy at a resonant frequency that is based on a size of a bubble.

14. The method of claim 12, wherein the ablation energy is adjusted based on an amplitude of the detected energy.

15. The method of claim 12, wherein the ablation energy is adjusted to maintain bubble diameters less than about 100 micrometers.

16. The method of claim 12, further comprising interrogating body tissue receiving ablation energy with a single interrogation ultrasound frequency.

17. The method of claim 12, further comprising interrogating body tissue receiving ablation energy with a band limited spread spectrum signal.

18. The method of claim 12, further comprising interrogating body tissue receiving ablation energy with an interrogation signal transmitted at a frequency differing from a frequency of the energy emitted by collapsing or shrinking bubbles in the body tissue.

19. The system of claim 9, wherein the frequency of the energy emitted by collapsing or shrinking bubbles in the body tissue comprises a plurality of harmonics or sub-harmonics of the interrogation frequency.
